(19) United States
(12) Patent Application Publication
(10) Pub. No.: US 2019/0000526 A1
(43) Pub. Date: Jan. 3, 2019

(54) RADIO FREQUENCY SLIMMING TREATMENT APPARATUS

(71) Applicant: Shenzhen Peninsula Medical Co., Ltd., Shenzhen (CN)

(72) Inventors: Xiaobing LEI, Shenzhen (CN); Hongbo LUO, Shenzhen (CN)

(73) Assignee: Shenzhen Peninsula Medical Co., Ltd., Shenzhen (CN)

(21) Appl. No.: 15/853,760

(22) Filed: Dec. 23, 2017

(30) Foreign Application Priority Data
Jun. 30, 2017 (CN) 201710531009.9

(51) Int. Cl. A61B 18/14; A61B 18/12; A61B 18/18

(52) U.S. Cl. CPC A61B 18/14; A61B 18/1206; A61B 18/18; A61B 2018/1467; A61B 2018/1407; A61B 2018/1435; A61B 2018/126

(57) ABSTRACT

A radio frequency slimming treatment apparatus includes a plurality of connection components and a plurality of radio frequency slimming treatment heads, each of which includes a main body of the treatment head, a plurality of radio frequency electrodes, an interface that is employed to connect the radio frequency source configured over the main body of the treatment head, a fixation surface configured at the end of the main body of the treatment head that is distal from the interface. The plurality of radio frequency electrodes are configured over the fixation surface and arranged in the form of an array. As a result, the current of the radio frequency slimming treatment head of the present disclosure is uniform, the temperatures of the different regions of the radio frequency electrodes are uniform and the penetration depth of the radio frequency current is deeper, and the lipolytic effect is significant.

### CROSS-REFERENCE TO RELATED APPLICATION

The present application claims priority to Chinese Patent Application No. 201710531009.9 filed on Jun. 30, 2017, the disclosure of which is hereby incorporated by reference in its entirety.

### BACKGROUND

Radio frequency slimming is the most advanced slimming technology most recently recommended by the International Society of Aesthetic Plastic Surgery. it can achieve permanent slimming effect, reshape the natural curve of the body. It's working principles are: fat is heated by radio frequency current, as a result, extra fat of different body regions becomes coagulated and necrotized, slimming body contouring is therefore achieved.

### SUMMARY

The present disclosure relates to the field of medical devices, and specifically relates to a radio frequency slimming treatment head and a radio frequency slimming treatment apparatus.

The inventors of the present disclosure have recognized that, radio frequency slimming treatment heads of existing technologies usually adopt one radio frequency electrode. Because the resistance of human skin is much larger than the resistance of the radio frequency electrode and because of the skin effect during the transmission of the radio frequency current, more radio frequency current can only transmit along the edge region of the radio frequency electrode. As a result, the density of the radio frequency current in the central region is much lower than that in the edge region. Therefore, in existing radio frequency slimming treatment heads, problems exist. For example, the radio frequency current is not uniform and the penetration depth of the radio frequency is small.

The main purpose of the present disclosure is to provide a radio frequency slimming treatment apparatus to improve the uniformity and penetration depth of the radio frequency current of radio frequency slimming treatment head.

In order to achieve the aforementioned purpose, in one aspect of the present disclosure, a radio frequency slimming treatment head is provided, which includes a main body of a treatment head; an interface that connects to a radio frequency source and is configured over the main body of the treatment head; a fixation surface configured at an end of the main body of the treatment head that is distal from the interface; and a plurality of radio frequency electrodes; wherein, the plurality of radio frequency electrodes are all configured over the fixation surface and arranged in the form of an array so that the density of the output current is uniform.

In some embodiments of the present disclosure, the plurality of radio frequency electrodes are all ring-shaped and take nesting arrangement from a center of the fixation surface.

In some embodiments, the radio frequency electrodes are circular ring-shaped or square ring-shaped.

In some embodiments, the plurality of radio frequency electrodes are connected in parallel.

In some embodiments, a plurality of electric contact points are configured over each radio frequency electrode and the plurality of electric contact points are arranged symmetrically to the center of the fixation surface.

In some embodiments, a treatment surface which is employed to contact the skin is formed at a side of the radio frequency electrodes that is distal from the fixation surface and a protecting film is configured over the treatment surface.

In some embodiments, the main body of the treatment head includes a mounting base with an accommodating chamber inside; an opening is configured at a side of the accommodating chamber that is distal from the interface; and a connection cover, which is snap fixed to the opening end of the mounting base; wherein the outer surface of the connection cover that is opposite to the opening forms the fixation surface.

In another aspect of the present disclosure, a radio frequency slimming treatment apparatus is provided, which includes a plurality of connection components and a plurality of radio frequency slimming treatment heads; wherein each one of the radio frequency slimming treatment heads is configured in one of the connection components; any adjacent radio frequency slimming treatment heads are connected through the connection components.

Wherein, the radio frequency slimming treatment head includes a main body of the treatment head; an interface that connects to a radio frequency source and is configured over the main body of the treatment head; a fixation surface configured at an end of the main body of the treatment head that is distal from the interface; and a plurality of radio frequency electrodes; wherein, the plurality of radio frequency electrodes are all configured over the fixation surface and arranged in the form of an array so that the density of the output current is uniform.

In some embodiments of the present disclosure, a mounting opening that is employed to install the radio frequency slimming treatment head is formed over the connection component; at least one pair of fastening lugs extend from the edge of the mounting opening towards the side of the radio frequency slimming treatment head; and the radio frequency slimming treatment head is clipped between the fastening lugs.

In some embodiments, sinking grooves that fit with the fastening lugs are configured over the outside wall of the main body of the treatment head; a bulge is configured at the bottom of each sinking groove; a hole that fits to the bulge is configured over each fastening lug.

In the first embodiment, the radio frequency slimming treatment apparatus is a monopolar radio frequency slimming treatment apparatus, which includes a radio frequency source including an anode output terminal and a cathode output terminal; a plurality of anode treatment handpieces connecting to the anode output terminal; wherein each anode treatment handpiece is provided with a radio frequency slimming treatment head; and a cathode plate connecting to the cathode output terminal.

In the second embodiment, the radio frequency slimming treatment apparatus is a bipolar radio frequency slimming treatment apparatus, which includes a radio frequency source including an anode output terminal and a cathode output terminal; at least one pair of treatment handpieces; each pair of the treatment handpieces includes an anode treatment handpiece connecting to the anode output terminal and a cathode treatment handpiece connecting to the cathode output terminal; and each anode treatment handpiece and cathode treatment handpiece is provided with a radio frequency slimming treatment head.

In some embodiments, the distance between the radio frequency slimming treatment heads of the anode treatment handpiece and the cathode treatment handpiece is 5 cm˜25 cm.

The radio frequency slimming treatment head according to some embodiments of the present disclosure includes a plurality of radio frequency electrodes that are configured over the fixation surface of the radio frequency slimming treatment head in the form of an array. Thus, the plurality of radio frequency electrodes are distributed regularly over the fixation surface, which improved the uniformity of the radio frequency current of the radio frequency slimming treatment head provided in the various embodiments of the present disclosure.

In addition, because the radio frequency current of neighboring radio frequency electrodes interact with one another, radio frequency current can transmit deeper along the vertical direction. Thus, the penetration depth of the radio frequency current is improved. Therefore, the radio frequency current of the radio frequency slimming treatment head in the various embodiments of the present disclosure is uniform, the temperature of different regions of the radio frequency electrodes is uniform, the penetration depth of the radio frequency current is deep, and the lipolytic effect is remarkable.

### BRIEF DESCRIPTION OF DRAWINGS

In order to clearly illustrate the embodiments in the present disclosure, the accompanying drawings that need to be used in the description of the embodiments will be introduced briefly. Apparently, the following accompanying drawings are just some embodiments of the present disclosure. For those skilled in the art, they can acquire other accompanying drawings based on structures shown in these accompanying drawings on the premise of not paying creative labor.

FIG. 1 is a structural diagram of a radio frequency slimming treatment head provided in some embodiments of the present disclosure;

FIG. 2 illustrates an exploded view of the radio frequency slimming treatment head of FIG. 1;

FIG. 3 illustrates another exploded view of the radio frequency slimming treatment head of FIG. 1;

FIG. 4 shows a structural diagram of the square ring-shaped radio frequency electrodes provided in some embodiments of the present disclosure;

FIG. 5 shows a structural diagram of the circular ring-shaped radio frequency electrodes provided in some other embodiments of the present disclosure;

FIG. 6 shows a diagram of the wiring of the radio frequency electrodes of FIG. 5;

FIG. 7 shows a diagram of the distribution of the electrical contact points of the radio frequency electrodes of FIG. 5;

FIG. 8 shows a diagram illustrating the working principle of the crowding effect of the radio frequency current of a plurality of radio frequency electrodes;

FIG. 9 shows a structural diagram after the radio frequency slimming treatment head is fixed to the connection component;

FIG. 10 shows a structural diagram of the radio frequency slimming treatment head disassembled from the connection component of FIG. 9;

FIG. 11 illustrates a front side view of a plurality of radio frequency slimming treatment heads connected through a plurality of connection components;

FIG. 12 illustrates a backside view of a plurality of radio frequency slimming treatment heads connected through a plurality of connection components;

FIG. 13 shows a structural diagram of a monopolar radio frequency slimming treatment apparatus provided in the first embodiment of the present disclosure;

FIG. 14 shows a structural diagram of a bipolar radio frequency slimming treatment apparatus provided in the second embodiment of the present disclosure; and

FIG. 15 shows a structural diagram of another bipolar radio frequency slimming treatment apparatus provided in the second embodiment of the present disclosure.

### DETAILED DESCRIPTION

With reference to the drawings of some embodiments disclosed herein, the technical solutions of the present disclosure will be described in a clear and fully understandable way. It is noted that the described embodiments are merely representative and are not all the embodiments of the present disclosure. Based on the example embodiments in the present disclosure, other embodiments that may be conceived by those of ordinary skill are in the protection scope of the present disclosure.

Various embodiments of the present disclosure provides a radio frequency slimming treatment head, which improves the uniformity and penetration depth of the radio frequency current.

In slimming treatments based on thermal effect, when the temperature of the fat layer exceeds 50° C. for 1 minute, the fat activity decreases 20%; when the temperature of the fat layer exceeds 45-50° C. for 3 minutes, the fat activity decreases 40%; when the temperature maintains at 43-45° C. for 15 minutes, the fat activity of decreases 40%. The pain threshold of skin is usually 43° C.

In traditional radio frequency slimming apparatus, a large piece of electrode is employed as the application component. Usually, the uniformity of the energy distribution of such radio frequency slimming apparatus is not good and the penetration depth is low. Low distribution uniformity may cause ineffective treatment in some regions, excessive treatment dose in some other regions, or even empyrosis. In some improved solutions, the method of outputting by a plurality of electrodes is adopted. As a result, the uniformity can be improved in a certain degree, however, the penetration depth is not adequately improved.

In the traditional large-size electrode solution, usually a large piece of electrode is employed as the output electrode. However, because of the skin effect of the transmission of the radio frequency current, and because the resistance of the human skin is significantly larger than the resistance of the radio frequency electrode, more radio frequency current transmits along the edge region of the radio frequency electrode, which in turn causes the density of the radio frequency current at the central region much lower than that in the periphery region. Therefore, the output of the radio frequency is not uniform and the current density is more concentrated at the periphery region of the electrode. According to Q=I*I*R*t, wherein, I is the current passing through the tissue, R is the resistance of the tissue, t is time, the thermal effect of the periphery region of the tissue is significantly larger than that in the central region in the treatment process.

Embodiments of the present disclosure adopt a plurality of radio frequency electrodes and the radio frequency current transmits along different radio frequency electrodes. Thus the distribution of the current is relatively uniform. According to Q=I*I*R*t, the energy distribution is therefore uniform. In the multiple electrodes solution adopted by the present disclosure, there can be 3, more than 3 electrodes, or even 5-20 electrodes. The radio frequency electrodes can be plate-shaped electrodes, ring-shaped electrodes or electrodes in other shapes, preferably, ring-shaped electrodes are adopted.

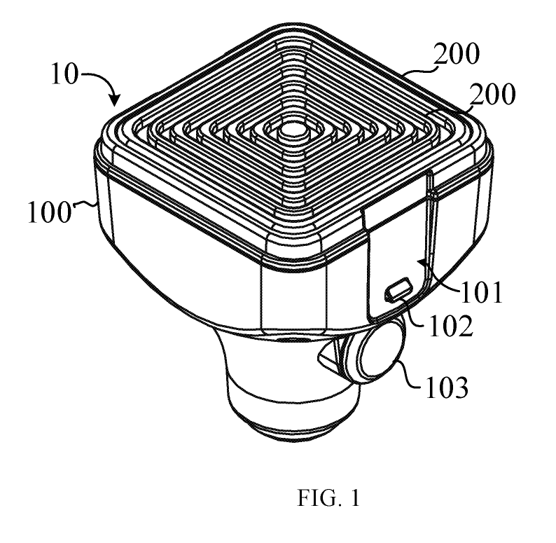

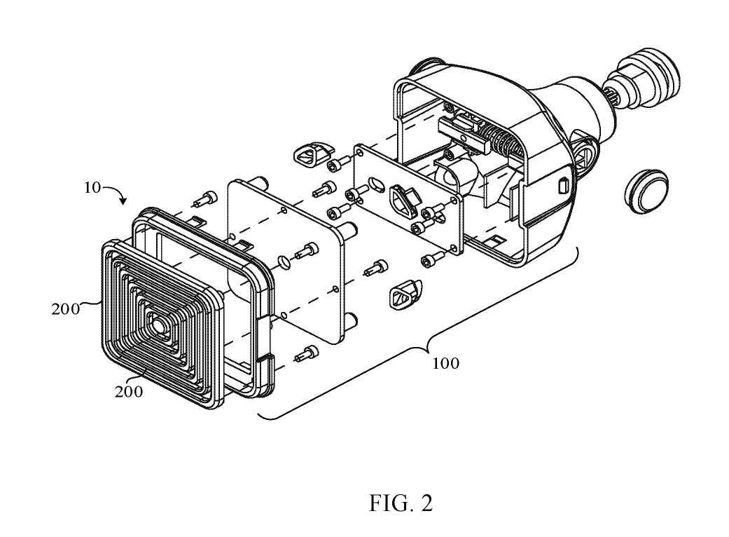

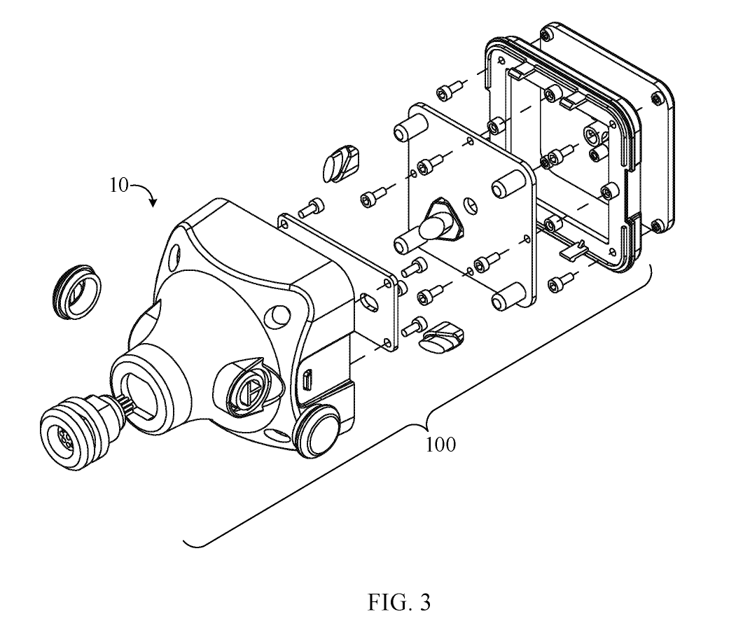

In some embodiments of the present disclosure, as shown in FIGS. 1-3, the radio frequency slimming treatment head includes a main body of a treatment head 100, an interface that connects to a radio frequency source and is configured over the main body of the treatment head 100, a fixation surface configured at an end of the main body of the treatment head 100 that is distal from the interface, a plurality of radio frequency electrodes 200 that are all configured over the fixation surface, and the plurality of radio frequency electrodes 200 are arranged in the form of an array so that the density of the output current is uniform.

Because the plurality of radio frequency electrodes 200 are arranged in the form of any array over the fixation surface of the main body of the treatment head 100, that is: the plurality of radio frequency electrodes 200 are arranged regularly over the fixation surface, the uniformity of the radio frequency current can be improved. In addition, because the radio frequency current of neighboring radio frequency electrodes 200 interacts with one another, the radio frequency current can transmit deeper in vertical direction. As a result, the penetration depth of the radio frequency current is improved. Therefore, the radio frequency current of the radio frequency slimming treatment head of the present disclosure is uniform, the temperature of different regions of the radio frequency electrodes 200 is uniform, the penetration depth of the radio frequency current is deeper, and the lipolytic effect is significant.

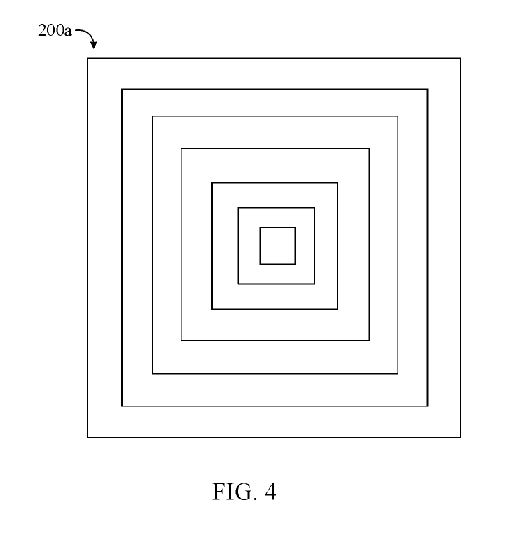

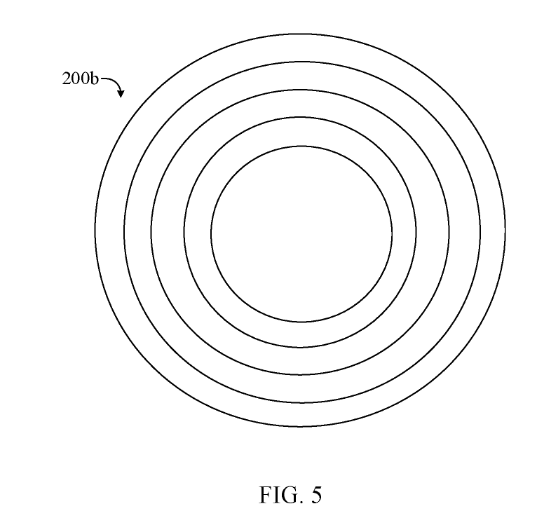

With reference to FIGS. 4-5, in order to further enhance the interaction among the radio frequency electrodes 200, in some embodiments of the present disclosure, the plurality of the radio frequency electrodes 200 are all ring-shaped and take nesting arrangement from a center of the fixation surface. Because the plurality of radio frequency electrodes 200 take nesting arrangement extending from the center to the periphery region, each electrode will not only interact with the neighboring electrodes in a greater degree but also interact with other electrodes in a less degree. Therefore, each electrode realizes its maximal interaction with other electrodes so that the radio frequency current is enhanced as much as possible and the penetration depth of the radio frequency current is improved.

In some embodiments of the present disclosure, the radio frequency electrodes 200 are circular ring-shaped or square ring-shaped. The circular ring-shaped radio frequency electrodes 200b and the square ring-shaped radio frequency electrodes 200a are beautifully shaped and easy to manufacture. However, the design of the present disclosure is not limited herein. In other embodiments, the radio frequency electrodes 200 can also be in the shape of plates. The array configuration of the radio frequency electrodes 200 is also not limited to nesting arrangement extending from center to periphery, but also they can take matrix node distribution with intervals. In addition, the number of the electrodes may be 2, more than 2, or 5˜20 but not limited to 5˜20.

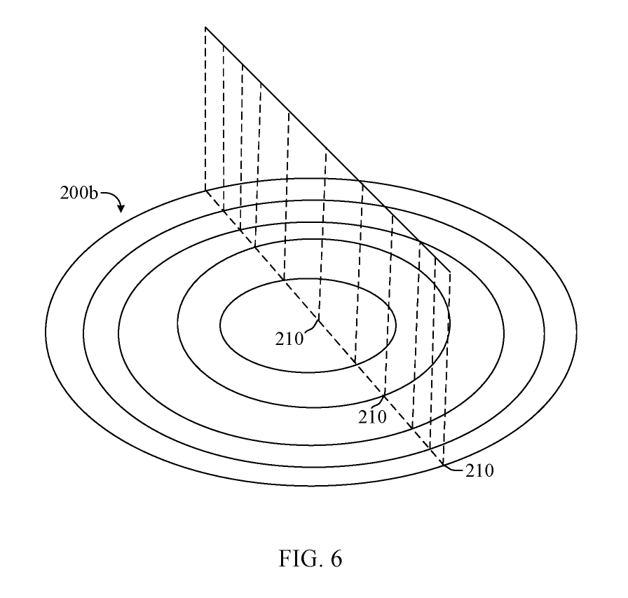

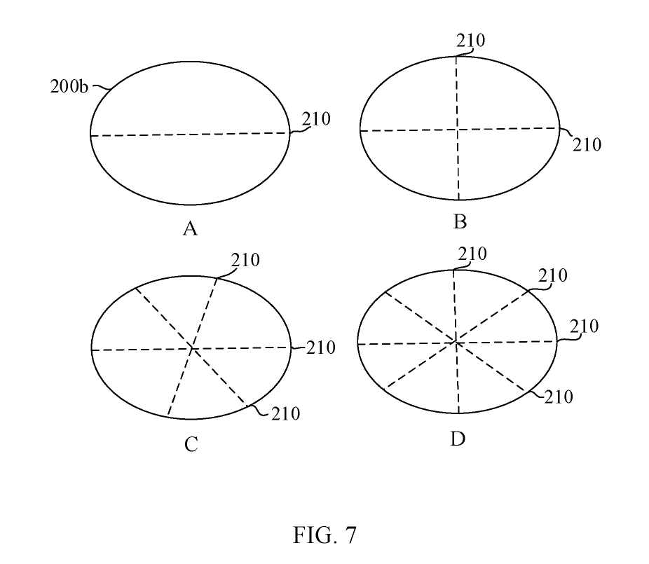

With reference to FIGS. 6-8, in order to further improve the uniformity of the radio frequency current, in some embodiments of the present disclosure, the plurality of radio frequency electrodes 200 are configured in parallel. The electrical potentials of the radio frequency electrodes 200 configured in parallel are equal and there is no difference in the electrical potential among the plurality of radio frequency electrodes 200. Therefore, the density of the radio frequency current is uniform.

In some embodiments of the present disclosure, a plurality of electric contact points 210 are configured over each radio frequency electrode 200. The plurality of electric contact points 210 are arranged symmetrically with reference to the center of the fixation surface. Because a plurality of electric contact points 210 are configured over each radio frequency electrode 200, the transmission path of the electric current along the radio frequency electrodes is relatively short. Thus the loss of electricity over the radio frequency electrodes 200 is relatively small and the radio frequency current can be kept to be relatively high in value and uniform in distribution.

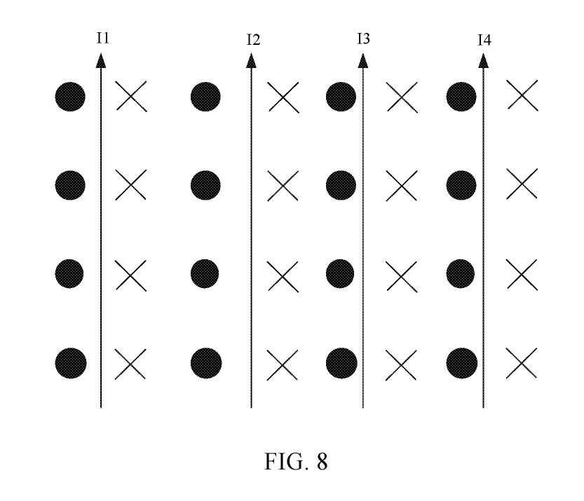

FIG. 8 shows a diagram illustrating the radio frequency current of several groups of independent multiple electrodes and the directions of the radio frequency current are the same. During the current transmission, electric current produced corresponding magnetic fields. Current I1 and current I2 are attracted to each other, current I2 and current I3 are attracted to each other, and current I3 and current I4 are attracted to each other. Therefore, the transmission of the current is concentrated and the focusing effect is resulted during transmission process. Especially for ring-shaped electrodes, when the polarity of the electrode are the same, there is a focusing effect at each angle in the ring-shaped electrodes. That is, the current I at a certain layer of the skin tissue is higher than that at other surrounding regions. According to Q=I*I*R*t, the thermal effect at the layer with higher current I is the most significant, therefore, the temperature increases the most. In addition, the heat dissipation capability of the central region is much lower than that of the edge region. Therefore, the temperature of the central region is much higher.

In some embodiments of the present disclosure, a treatment surface which is employed to contact the skin is formed at the side of the radio frequency electrodes 200 that is distal from the fixation surface. A protecting film is configured over the treatment surface. The function of the treatment surface is to eliminate the feeling of asperity that would exists when the electrodes are in contact with the skin directly; improve the comfortability of the treatment process; avoid the discharge phenomenon at the edge of the radio frequency electrodes so that the heat radiated from the treatment surface can be uniform.

In some embodiments, the protecting film are produced with any one of the following insulating material such as rubber, ceramic, plastic, polymer or paint; when the protecting film is produced with material such as rubber, ceramic, plastic, polymer, the protecting film is adhered and fixed to the treatment surface; when the protecting film is produced with paint, the protecting film is painted and fixed to the treatment surface. Therefore, the fixation methods of the protecting film can be selected according to the material of the protecting film. It will not be repeated herein.

With reference to FIGS. 1-3, the structure of the main body of the radio frequency slimming treatment head according to some embodiments will be further described in detail. The main body of the radio frequency slimming treatment head 100 includes a mounting base (not marked in figures); a connection cover (composed of a plurality of components in the figures, not marked in the figures); an accommodating chamber configured inside the mounting base; a PCB circuit board configured inside the accommodating chamber that connects to a power source interface; an opening formed at the side of the accommodating chamber that is distal from the power source interface; wherein the connection cover is snap fixed to the end of the mounting base where the opening is located; the outer surface of the connection cover that is opposite to the opening forms the fixation surface that is employed to configure the radio frequency electrodes 200.

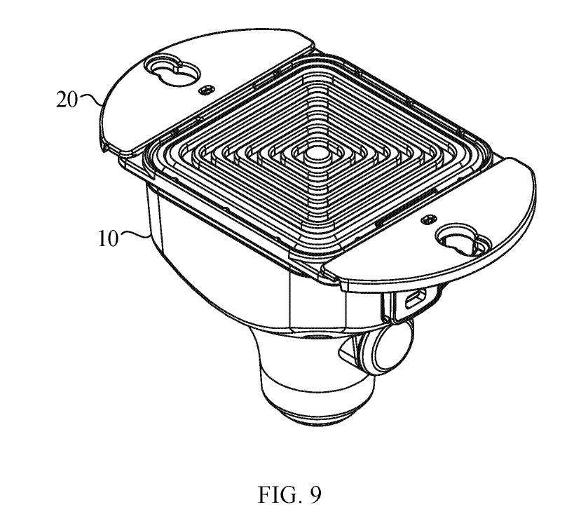

With reference to FIGS. 9-11, the present disclosure further provides a radio frequency slimming treatment apparatus. The radio frequency slimming treatment apparatus includes a plurality of connection components 20 and a plurality of radio frequency slimming treatment heads 10. Each radio frequency slimming treatment head 10 is installed in a connection component 20. Neighboring radio frequency slimming treatment heads 10 are connected through the connection components 20. For the specific structure of the radio frequency slimming treatment heads 10, please refer to the aforementioned embodiments. Because the radio frequency slimming treatment apparatus of the present disclosure adopts all technical solutions of the aforementioned embodiments, it at least has all the beneficial effects of the technical solutions of the aforementioned embodiments, which will not be repeated herein.

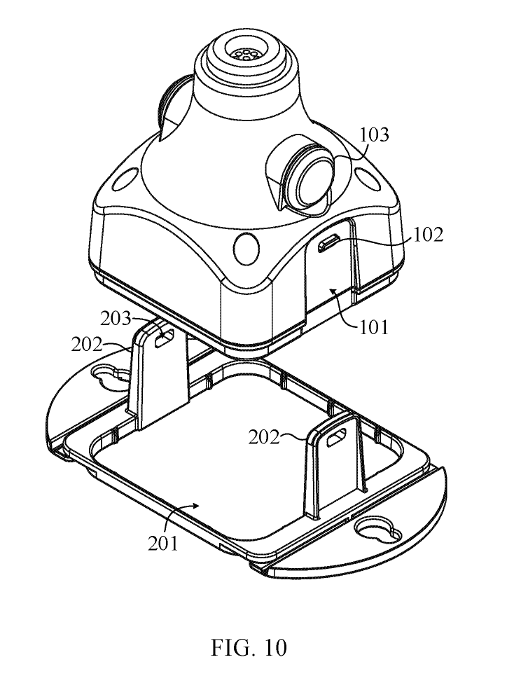

In some embodiments of the present disclosure, with reference to FIG. 10, a mounting opening 201 which is employed to mount the radio frequency slimming treatment head 10 is formed over the connection component 20. At least one pair of fastening lugs 202 is extended from the edge of the mounting opening 201 to the side of the radio frequency slimming treatment head 10. The radio frequency slimming treatment head 10 is clipped between the fastening lugs 202. Each radio frequency slimming treatment head 10 is connected to other radio frequency slimming treatment heads 10 through one connection component 20, so that the radio frequency slimming treatment heads 10 of the radio frequency slimming treatment apparatus can be configured in a modular fashion. As such, it is easy to maintain and repair. Because the radio frequency slimming treatment apparatus has a plurality of radio frequency slimming treatment heads 10, the treatment region of the radio frequency slimming treatment apparatus is increased.

In order to ensure the mounting strength of the radio frequency slimming treatment head 10 and the connection component 20, sinking grooves 101 that fit to the fastening lugs 202 are configured over the outer wall of the main body of the treatment head 100. A bulge 102 is configured at the bottom of each sinking groove 101. A hole that fits to the bulge 102 is configured over each fastening lug 202. As a result, after the radio frequency treatment head 10 is mounted with the connection component 20, the fastening lugs 202 of the connection components 20 are mounted into the sinking grooves 101 over the outer wall of the main body of the radio frequency slimming treatment head 100 and the bulges 102 inside the sinking grooves 101 are snap fixed to the holes over the fastening lugs 202. As a result, the radio frequency slimming treatment head 10 and the connection component 20 are secured. There is a bondage opening at the edge of the connection component 20, which is employed for bondage connection. The treatment head is bonded and fixed to the body through the bondage.

In some embodiments of the present disclosure, the radio frequency slimming treatment head 10 further includes a button 103, which can control the location of the bulge, so that the bulge can sink into the bottom of the sinking groove 101 or protrude from the bottom of the sinking groove 101. As a result, it is more convenient to mount or unmount the radio frequency slimming treatment head 10 and the connection component 20.

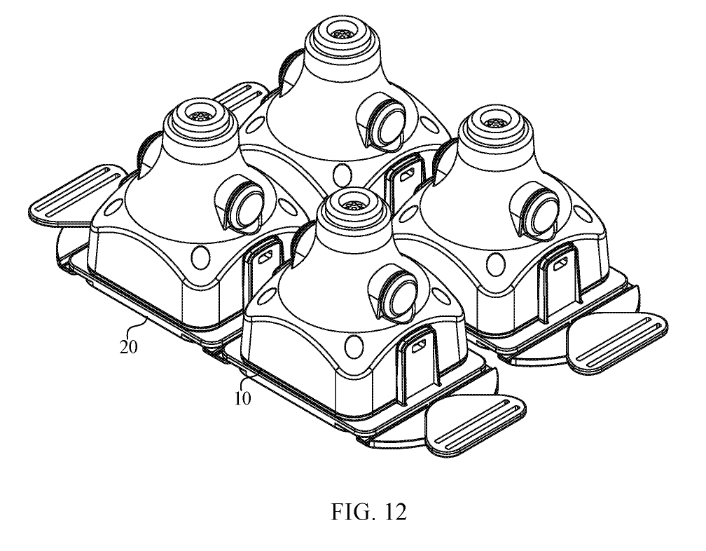

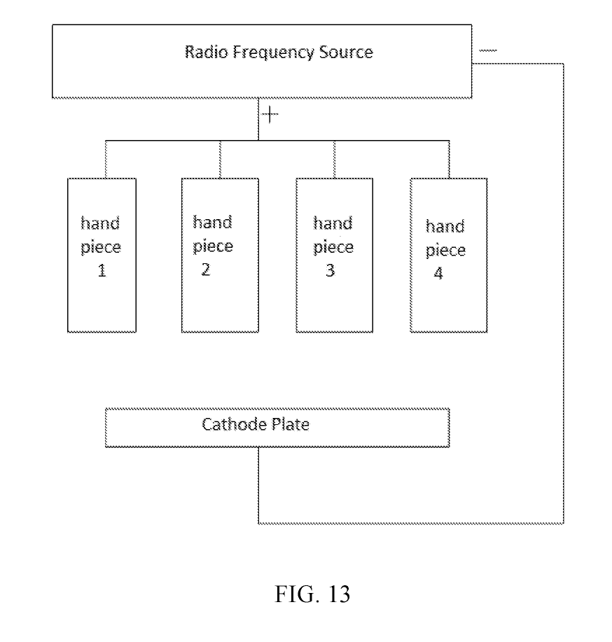

With reference to FIGS. 12 and 13, a first embodiment of the present disclosure is a monopolar radio frequency slimming treatment apparatus, which has deep penetration depth of the radio frequency current. The monopolar radio frequency treatment apparatus includes a radio frequency source, a cathode plate and a plurality of anode treatment handpieces, wherein, the radio frequency source has an anode output terminal and a cathode output terminal; the plurality of anode treatment handpieces are connected to the anode output terminal, and the radio frequency slimming treatment head 10 is configured over each of the plurality of anode treatment handpieces; the cathode plate is connected to the cathode output terminal.

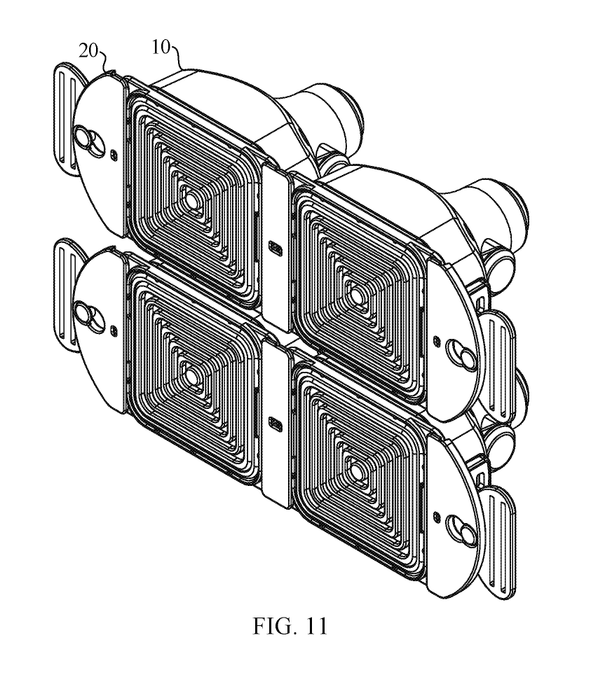

The number of the plurality of treatment handpieces can specifically be 1 or more than 1. For example, in the combination of 2*1, 3*1, 4*1, 5*1, 6*1, 2*2, 2*3, 2*4, 2*5 and so on. The 2*2 combination scheme is adopted in FIG. 11 and FIG. 12. The corresponding connection components 20 can have different specifications, which are adopted to suit different combination schemes.

Different combination of the treatment heads can be applied to the treatment of different parts of the body. For example, for abdomen, the 2*4 combination can be adopted; for arms, the 2*1 combination can be adopted. This kind of combination scheme can greatly improve the speed of the treatment and reduce the difficulties of operation for medical doctors.

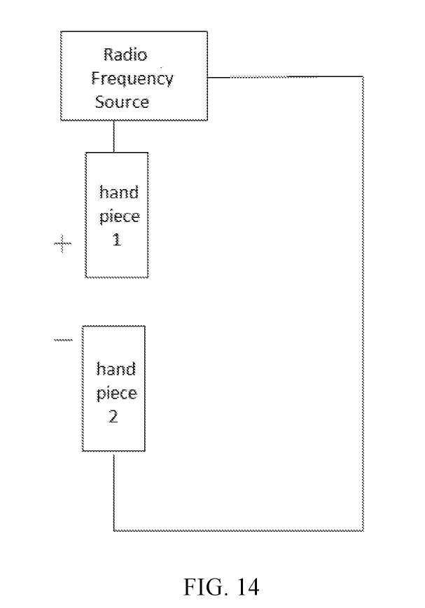

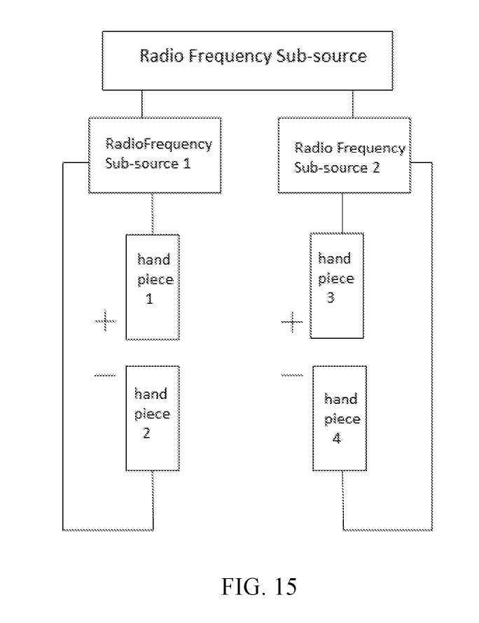

With reference to FIG. 14 and FIG. 15, the second embodiment of the radio frequency slimming treatment apparatus is a bipolar radio frequency slimming treatment apparatus, which has not only anode treatment handpieces but also cathode treatment handpieces. Therefore, the utilization rate of the electrodes is high. The bipolar radio frequency slimming treatment apparatus of FIG. 14 includes a radio frequency source and a pair of treatment handpieces, wherein, the radio frequency source has an anode output terminal and a cathode output terminal; the bipolar radio frequency slimming treatment apparatus of FIG. 15 is configured with two radio frequency sub-sources, each radio frequency sub-source is configured with a pair of treatment handpieces; each pair of the treatment handpieces includes an anode treatment handpiece connected to the anode output terminal and a cathode treatment handpiece connected to the cathode output terminal. The radio frequency slimming treatment heads 10 are configured over both the anode treatment handpieces and the cathode treatment handpieces.

In order to avoid the interference between the anodes and the cathodes and ensure the treatment depth of the radio frequency current, the range of the values of the distance between the radio frequency slimming treatment head 10 of the anode treatment handpiece and the radio frequency slimming treatment head 10 of the cathode treatment handpiece is 5 cm to 25 cm.

Although specific embodiments have been described above in detail, the description is merely for purposes of illustration. It should be appreciated, therefore, that many aspects described above are not intended as required or essential elements unless explicitly stated otherwise. Various modifications of, and equivalent acts corresponding to, the disclosed aspects of the exemplary embodiments, in addition to those described above, can be made by a person of ordinary skill in the art, having the benefit of the present disclosure, without departing from the spirit and scope of the disclosure defined in the following claims, the scope of which is to be accorded the broadest interpretation so as to encompass such modifications and equivalent structures.

1. A radio frequency slimming treatment head, comprising:
a main body of the treatment head;
an interface that is employed to connect a radio frequency source and is configured over the main body of the treatment head;
a fixation surface that is configured at the side of the main body of the treatment head that is distal from the interface; and
a plurality of radio frequency electrodes that are all configured over the fixation surface, and are arranged in the form of an array to ensure the density of the output current to be uniform.

2. The radio frequency slimming treatment head of claim 1, wherein:
the plurality of radio frequency electrodes are all ring-shaped; and
the plurality of radio frequency electrodes take nesting arrangement from the center of the fixation surface.

3. The radio frequency slimming treatment head of claim 2, wherein the radio frequency electrodes are circular ring-shaped or square ring-shaped.

4. The radio frequency slimming treatment head of claim 3, wherein the plurality of radio frequency electrodes are connected in parallel.

5. The radio frequency slimming treatment head of claim 4, wherein:
a plurality of electric contact points are configured over each radio frequency electrode; and
the plurality of electric contact points are arranged symmetrically to the center of the fixation surface.

6. The radio frequency slimming treatment head of claim 1, wherein the treatment surface that is employed to contact the skin is formed at a side of the radio frequency electrodes that is distal from the fixation surface; and
a protecting film is disposed over the treatment surface.

7. The radio frequency slimming treatment head of claim 1, wherein, the main body of the treatment head comprises:
a mounting base;
an accommodating chamber configured in the mounting base;
an opening configured at a side of the accommodating chamber that is distal from the interface; and
a connection cover, which is snap fixed to the opening of the mounting base, wherein, the outer surface of the connection cover that is opposite to the opening forms the fixation surface.

8. A radio frequency slimming treatment apparatus, comprising:
a plurality of connection components; and
a plurality of radio frequency slimming treatment heads;
wherein each of the plurality of radio frequency slimming treatment heads comprises:
a main body of the treatment head;
an interface that is employed to connect a radio frequency source and is configured over the main body of the treatment head;
a fixation surface that is configured at the side of the main body of the treatment head that is distal from the interface; and
a plurality of radio frequency electrodes that are all configured over the fixation surface, and are arranged in the form of an array to ensure the density of the output current to be uniform;
wherein each of the radio frequency slimming treatment heads is configured in one of the connection components; and
all neighboring radio frequency slimming treatment heads are connected through the connection components.

9. The radio frequency treatment apparatus of claim 8, further comprising:
a mounting opening that is employed to install the radio frequency slimming treatment head and is formed over the connection component;
at least one pair of fastening lugs extending from the edge of the mounting opening towards the side of the radio frequency slimming treatment head;
wherein the radio frequency slimming treatment head is clipped between the fastening lugs.

10. The radio frequency slimming treatment apparatus of claim 9, further comprising:
sinking grooves that fit to the fastening lugs over the outer wall of the main body of the treatment head;
a bulge configured at the bottom of each sinking grooves; and
a hole that fits to the bulge over each fastening lug.

11. The radio frequency slimming treatment apparatus of claim 8, wherein the apparatus is a monopolar radio frequency slimming treatment apparatus comprising:
a radio frequency source, which comprises an anode output terminal and a cathode output terminal;
a plurality of anode treatment handpieces, which are connected to the anode output terminal; wherein, the radio frequency slimming treatment heads are configured over all anode treatment handpieces; and
a cathode plate, which is connected to the cathode output terminal.

12. The radio frequency slimming treatment apparatus of claim 8, wherein the apparatus is a bipolar radio frequency slimming treatment apparatus comprising:
a radio frequency source, which comprises an anode output terminal and a cathode output terminal; and
at least one pair of treatment handpieces;
wherein each pair of treatment handpieces comprise an anode treatment handpiece that is connected to the anode output terminal and a cathode treatment handpiece that is connected to the cathode output terminal; and
the radio frequency slimming treatment heads are configured over both the anode treatment handpiece and the cathode treatment handpiece.

13. The radio frequency slimming treatment apparatus of claim 12, wherein a distance between the radio frequency slimming treatment head of the anode treatment handpiece and the radio frequency slimming treatment head of the cathode treatment handpiece is 5 cm˜25 cm.

14. The radio frequency treatment apparatus of claim 8, wherein:
the plurality of radio frequency electrodes are all ring-shaped; and
the plurality of radio frequency electrodes take a nesting arrangement from the center of the fixation surface.

15. The radio frequency treatment apparatus of claim 8, wherein the radio frequency electrodes are circular ring-shaped or square ring-shaped.

16. The radio frequency treatment apparatus of claim 8, wherein the plurality of radio frequency electrodes are connected in parallel.

17. The radio frequency treatment apparatus of claim 8, wherein:
a plurality of electric contact points are configured over each radio frequency electrode; and
the plurality of electric contact points are arranged symmetrically to the center of the fixation surface.

18. The radio frequency treatment apparatus of claim 8, wherein:
the treatment surface that is employed to contact the skin is formed at the side of the radio frequency electrodes that is distal from the fixation surface; and
a protecting film is configured over the treatment surface.

19. The radio frequency treatment apparatus of claim 8, wherein the main body of the treatment head comprises:
a mounting base;
an accommodating chamber configured in the mounting base;
an opening configured at a side of the accommodating chamber that is distal from the interface; and
a connection cover, which is snap fixed to the opening of the mounting base, wherein, the outer surface of the connection cover that is opposite to the opening forms the fixation surface.
